(19) United States
(12) Patent Application Publication
(10) Pub. No.: US 2025/0318918 A1
(43) Pub. Date: Oct. 16, 2025

(54) STENTS AND PROCEDURES FOR PLACEMENT THEREOF

(71) Applicant: Charles Winston Weisse, New York, NY (US)

(72) Inventor: Charles Winston Weisse, New York, NY (US)

(21) Appl. No.: 19/096,202

(22) Filed: Mar. 31, 2025

Related U.S. Application Data
(60) Provisional application No. 63/570,892, filed on Mar. 28, 2024.

(51) Int. Cl. A61F 2/04; A61F 2/95

(52) U.S. Cl. CPC A61F 2/04; A61F 2/95; A61F 2002/046; A61F 2002/9534; A61F 2220/0008; A61F 2230/0008; A61F 2250/0039; A61F 2250/0059; A61F 2250/0081; A61F 2250/0082

(57) ABSTRACT

Self-expanding, tubular (tapered or constant diameter), oval or elliptical (not circular) airway stents suitable for placement under open or minimally invasive approaches within patients of various sizes and of various species, and methods for their placement in a tracheobronchial tree of a patient. The airway stent may be provided in various lengths and have an oval or elliptical cross-sectional shape that is either constant or variable in both short axis dimensions. The airway stents are particularly suited for treating patients with large airway obstructions in whom the natural airway cross-sectional dimensions are oval or elliptical rather than circular. The airway stent allows for more anatomic filling of the natural airway lumen, reduces the need to oversize the airway stent diameter and therefore reduces subsequent stent fatigue and fracture, and improves stent-tracheal wall contact to minimize gaps that lead to mucus trapping and chronic infections with subsequent tissue ingrowth and airway obstruction.

### CROSS-REFERENCE TO RELATED APPLICATIONS

This application claims the benefit of U.S. Provisional Application No. 63/570,892 filed Mar. 28, 2024, the contents of which are incorporated herein by reference.

### BACKGROUND OF THE INVENTION

The present invention generally relates to stents and procedures for placing stents in patients, including but not limited to animal patients that have tracheobronchial collapse or narrowing. More particularly, this invention relates to airway stents and procedures for implanting such a stent, for example, by placing the airway stent under fluoroscopic, radiographic, endoscopic, or surgical observation.

Canine Tracheal Collapse Syndrome (CTCS) is a common and clinically devastating benign condition affecting mainly toy- and small breed dogs of all ages. There are several underlying causes of this condition that have not been fully elucidated, however diagnosis is straightforward based upon patient signalment, clinical signs, and radiographic, fluoroscopic, endoscopic, and/or cross-sectional imaging. Traditional medical management includes anti-inflammatories antitussives, and antibiotics, however between 30% and 85% of patients (depending on underlying cause) will ultimately fail medical management within a median of 167 days and a median survival time of 3.7 years without further intervention. Traditional surgical treatments have included extraluminal prostheses placed around the trachea for external support. However, only the cervical trachea is amenable to this option (access to thoracic trachea and bronchi is limited), and complications are common including laryngeal paralysis, collapse beyond the rings, airway obstruction, and death.

Minimally invasive treatments have nearly replaced open surgical procedures with the placement of an airway stent (which as used herein includes what are commonly referred to as tracheal or tracheobronchial stents) delivered through the airway under fluoroscopic, radiographic, or endoscopic guidance without the need for open surgery. These techniques provide similar results to surgery without the associated complications (morbidities), allow treatment of very debilitated patients that would otherwise not likely survive surgery, and permit treatment of the entire trachea as well as bronchi. Current airway stents have solved some of the problems with previous techniques for providing a patent airway. These devices have been reported to function well with minimal complications compared to traditional open surgery when medical management has failed.

The most common complications encountered have included infection (approximately 66%), tissue ingrowth (approximately 19-38%), and stent fracture (approximately 25%). Stent fracture is associated with inappropriate stent sizing to an often nonuniform-shaped tracheobronchial tree (FIGS. 1A-1C) (Violette et al. 2019). Tissue ingrowth has several underlying causes but is most often due to mucus trapping between the stent and airway wall because a “round” stent (which as used herein refers to a stent with a circular cross-sectional shape) does not fully conform to the airway lumen, creating one or more spaces (hereinafter, “gaps”) between the stent and airway wall. Such a gap prevents endothelialization of the stent within the airway wall. Subsequent mucus accumulation leads to infection with tissue proliferation and airway occlusion (FIGS. 2A-2F and 3A-3E). To avoid airway gaps, some operators oversize the stent further to limit gaps, however this increases the likelihood of subsequent stent fatigue and fracture (Weisse et al. 2019, Violette et al. 2019).

Recently, the development of tapered airway stents has improved stent fit to tracheal walls by reducing the amount of stent oversizing within the intra-thoracic trachea. This has likely reduced the number of subsequent stent fractures, however tapered stents have round (i.e., circular) cross-sections and can still result in gaps, either leading to mucus accumulation or still requiring stent oversizing with the predictable deleterious results described above.

It is clear there is an ongoing need for devices that can treat various causes of airway collapse, narrowing, or compression, regardless of etiology, patient species, or size, in a rapid, simple, safe, and effective manner. There is a need for an airway stent capable of overcoming the shortcomings of the prior art, particularly fracture, infection, or tissue ingrowth.

### BRIEF DESCRIPTION OF THE INVENTION

The intent of this section of the specification is to briefly indicate the nature and substance of the invention, as opposed to an exhaustive statement of all subject matter and aspects of the invention. Therefore, while this section identifies subject matter recited in the claims, additional subject matter and aspects relating to the invention are set forth in other sections of the specification, particularly the detailed description, as well as any drawings.

The present invention provides, but is not limited to, self-expanding, tubular (tapered or constant diameter), oval or elliptical (meaning not circular) airway stents (hereinafter, sometimes referred to as “oval airway stent(s)” “oval or elliptical airway stent(s)” or more simply “airway stent(s)”) suitable for placement under open or minimally invasive approaches within patients of any size and of various species, and to methods for their placement in a tracheobronchial tree of a patient. The airway stent may be provided in various lengths and have an oval or elliptical (not circular) cross-sectional shape that is either constant or variable in both short axis dimensions.

According to a nonlimiting aspect of the invention, a procedure is provided for placing an airway stent for treating large airway (trachea and bronchus) obstructions in which the airway cross-section is oval or elliptical (not circular). The procedure includes providing an airway stent that has an oval or elliptical (not circular) cross-sectional shape, placing the airway stent in the airway through a delivery system comprising an endotracheal tube preplaced in a patient, ensuring positioning of the airway stent via delivery system markers indicating dorsoventral (anterior-posterior) positioning, partially deploying the airway stent under fluoroscopic guidance, repositioning the airway stent as needed through delivery system reconstrainment, completely deploying the airway stent so that the airway is patent, and removing of the delivery system through the airway.

According to another nonlimiting aspect of the invention, an airway stent for placement in an airway has an oval or elliptical cross-sectional shape such that the airway stent has different dorsoventral (anterior-posterior) diameter and lateral diameter (not circular).

A technical effect of the invention is that the airway stent is preferably capable of use in the treatment of many, if not all, causes of tracheobronchial obstruction, regardless of patient size or species. The airway stent may be used for airway strictures as well as intrinsic or extrinsic airway obstructions (benign or malignant neoplasia, for example).

Other aspects and advantages of this invention will be appreciated from the following detailed description.

### BRIEF DESCRIPTION OF THE DRAWINGS

FIGS. 1A through 1C are a series of radiographs in different canine patients with different types of round (i.e., circular) airway stents. The natural tapered diameter of the canine trachea requires oversizing of the airway stent diameter to completely fill the nonuniform, more oval or elliptical (not circular) tracheal cross-section. This results in dramatic stent oversizing in the intrathoracic trachea, additional stent fatigue and cycling in this location, and subsequent dorsocaudal stent fracture as demonstrated by the black arrows.

FIGS. 2A through 2F are a series of images of patients with a variety of round (circular) airway stents in the more oval canine trachea. FIG. 2A is an axial CT image of a dog with a round airway stent demonstrating incomplete filling of the oval tracheal lumen leaving gaps (black arrows) between the airway stent and tracheal wall. FIG. 2B is a lateral thoracic fluoroscopic image of a dog with a round airway stent demonstrating a visible “gap” (black arrows) between the airway stent tracheal wall due to the more oval tracheal diameter. FIGS. 20, 2D, 2E, and 2F are endoscopic tracheal images of four different dogs with round airway stents in oval tracheas demonstrating gaps (black arrows) between the airway stent and tracheal wall filling with mucus, which leads to subsequent mucus accumulation (white purulent material in images), infection, tissue ingrowth, and tracheal lumen obstruction.

FIGS. 3A through 3E are serial radiographic (FIGS. 3A, 3B, and 3C) or endoscopic (FIGS. 3D and 3E) images of different dogs with Canine Tracheal Collapse Syndrome (CTCS) and round airway stent placement. In the radiographic images, tissue ingrowth is apparent radiographically as white radio-opaque tissue (black arrows) growing into the black tracheal lumen. In the endoscopic images, tissue ingrowth into the tracheal lumen (black arrows) has occurred where there are gaps present due to the incomplete round stent-to-wall contact.

FIGS. 4A through 4F are axial CT images of three different canine patients (Patient 1 in FIGS. 4A and 4B, Patient 2 in FIGS. 4C and 4D, and Patient 3 in FIGS. 4E and 4F) with CTCS. FIGS. 4A, 4C, and 4E show cervical tracheal diameters, and FIGS. 4B, 4D, and 4F show intrathoracic tracheal diameters. Patient 1 (FIGS. 4A and 4B) demonstrates tracheal diameter ratios between 1:1.2 (cervical) and 1:2.6 (intrathoracic). Patient 2 (FIGS. 4C and 4D) demonstrates tracheal diameter ratios between 1:2 (cervical) and 1:1.8 (intrathoracic). Patient 3 (FIGS. 4E and 4F) demonstrates tracheal diameter ratios between 1:3 (cervical) and 1:1.8 (intrathoracic).

FIGS. 5A through 5D are three-dimensional (3D) renderings of an airway stent according to a nonlimiting embodiment of the invention. As represented in FIG. 5D, the airway stent is a non-round stent with differing dorsoventral (anterior-posterior) diameter (X) and] lateral diameter (Y=1.2X to 3X).

FIG. 6A is a 3D rendering of a round airway stent of the prior art, FIG. 6B is an image showing the cross-sectional shape of a canine trachea suffering from tracheal collapse, and FIG. 6C shows the round airway stent of FIG. 6A superimposed on the trachea of FIG. 6B, visualizing issues associated with placing a round stent in the non-round trachea of FIG. 6B, both during placement as well as long term implications. The inability of the round airway stent to completely fill the lumen of a collapsed trachea leads to gaps between the stent and the tracheal lumen, evidencing the degree of stent oversizing necessary to better fill a collapsed (non-round) trachea.

FIG. 6D is a 3D rendering of an airway stent according to a nonlimiting embodiment of the invention, FIG. 6E is the same image as FIG. 6B showing the cross-sectional shape of a collapsed canine trachea, and FIG. 6F shows the airway stent of FIG. 6D superimposed on the trachea of FIG. 6E, evidencing the improved conformal shape of the airway stent to the trachea and the ability of the airway stent to completely fill the lumen of the oval collapsed trachea of FIG. 6E. The lack of gaps between the airway stent and the collapsed tracheal lumen evidences that the airway stent does not require dramatic oversizing to fill the oval tracheal lumen. Similar to the airway stent of FIG. 5D, the airway stent is represented in FIGS. 6D and 6F as having a longer lateral diameter than its dorsoventral (anterior-posterior) diameter (e.g., Y=1.2X to 3X).

### DETAILED DESCRIPTION OF THE INVENTION

The intended purpose of the following detailed description of the invention and the phraseology and terminology employed therein is to describe what is shown in the drawings, which include the depiction of one or more nonlimiting embodiments of the invention, and to describe certain but not all aspects of the embodiment(s) depicted in the drawings. The following detailed description also identifies certain but not all alternatives of the depicted embodiment(s). Therefore, the appended claims, and not the detailed description, are intended to particularly point out subject matter regarded as the invention, including certain but not necessarily all the aspects and alternatives described in the detailed description.

Nonlimiting embodiments of airway stents are depicted in the drawings, along with conventional round airway stents for comparison. The airway stents may be referred to as oval tracheobronchial stents (OTS), and have what can be described as oval or elliptical (not circular) cross-sectional shapes for treating causes of airway obstruction. The airway stents may be used in animals, regardless of etiology, species, or patient size, and can be optimized for a given patient. It is also foreseeable that the airway stents may be used in humans. The airway stents disclosed herein are intended to allow for secure and facile placement of the stent within a patient, as will be evident from the following discussion. While the following discussion will sometimes refer to the stents as oval and/or elliptical airway stents and describe the placement of the stents in airways, stents within the scope of the invention are not limited to a particular oval or elliptical shape or placement in airways and instead can be used in any lumen that has an oval or elliptical (not circular) cross-section.

For purposes of placement in a lumen, stents described herein and their oval or elliptical (not circular) shapes are preferably specifically configured for placement based upon serial three-dimensional measurements of the lumen. As such, for purposes of placement in an airway within the tracheobronchial tree of a patient, the stents and their oval or elliptical shapes are configured for placement based upon serial three-dimensional measurements of the airway, a particular but nonlimiting example being a collapsed canine trachea. Such an airway stent can be tubular or tapered along its length and designed to maintain its position within the airway via a range of suitable sizes such that the airway stent can be slightly oversized to prevent migration while circumferentially opposing the airway wall without gaps between the airway stent and airway wall.

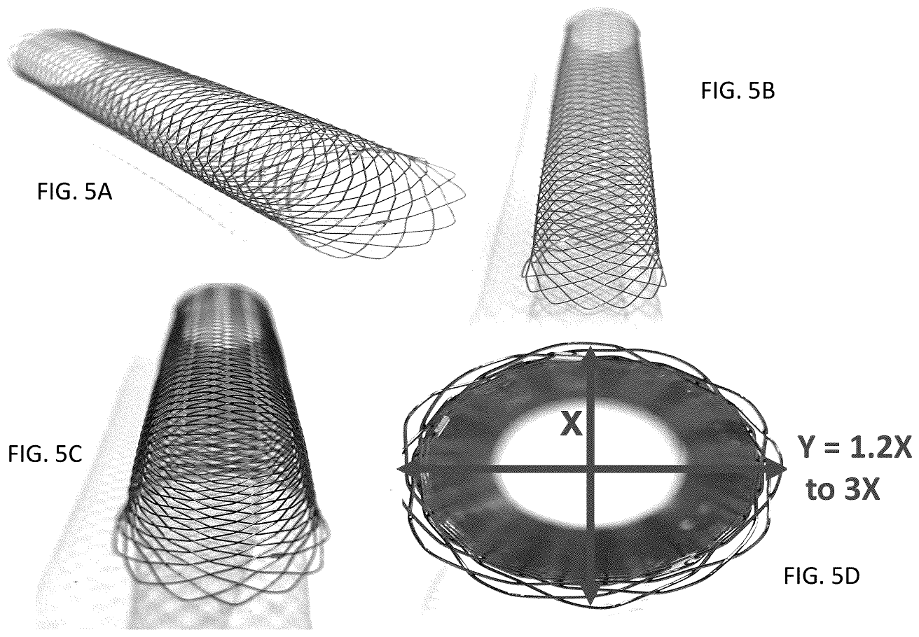

FIGS. 5A through 5D represent a first nonlimiting embodiment of an airway stent based on a self-expanding, wire-wound (mesh) design. It should be evident that the shape of the airway stent is configured differently from prior art round airway stents. Suitable dimensions of the airway stent will vary based upon collapse/obstruction length, type of tracheal collapse present, patient size, and conformation of the trachea (e.g., based upon previously measured CTCS patients). As nonlimiting examples, dorsoventral diameters and lateral diameters will be different to make the stent noncircular (“non-round”) in cross-sectional shape, resulting in the stents having an oval or elliptical cross-sectional shape. Sample dimensions could range from about 6 to about 20 mm and lateral diameters from about 8 to about 60 mm. These dimensions and ratios between the dorsoventral (short-axis/semi-minor) and lateral diameters (long-axis/semi-major) could also vary along the length of the airway, resulting in the desire for tubular or tapered stents or stents of non-uniform diameter throughout the length of the stent. The lengths of the airway stents may also vary; nonlimiting examples of stent lengths may range from about 15 to about 150 mm. The stents may be manufactured in both bare and covered (partial or complete) designs.

During placement of an airway stent, the stent may be premounted onto a standard (pin/pull) sheath delivery system or be manually mounted at the time of placement onto a sheath-type delivery system provided to the operator. Either delivery system type is preferably labelled clearly to delineate the appropriate positioning of the airway stent and delivery system within the trachea prior to deployment of the stent to ensure the stent is deployed in the correct position with the narrower diameter in a dorsoventral position.

A preferred procedure for placing the airway stent within a patient is to use a minimally invasive approach using a delivery system, such as through a preplaced endotracheal tube or through the open airway without surgery. Either delivery system permits non-invasive stent placement within the trachea. Deployment may be monitored fluoroscopically, endoscopically, radiographically, or via open surgery. Alternatively, an unmounted stent (or a deployed premounted stent) may be placed into the trachea through an open surgical approach, such as by passing the delivery system directly through the glottis, a tracheotomy, or other open surgical approach, to permit access to stabilize the airway stent with a suture or toggle as may be determined by the surgeon, as a nonlimiting example, in the case of a covered stent.

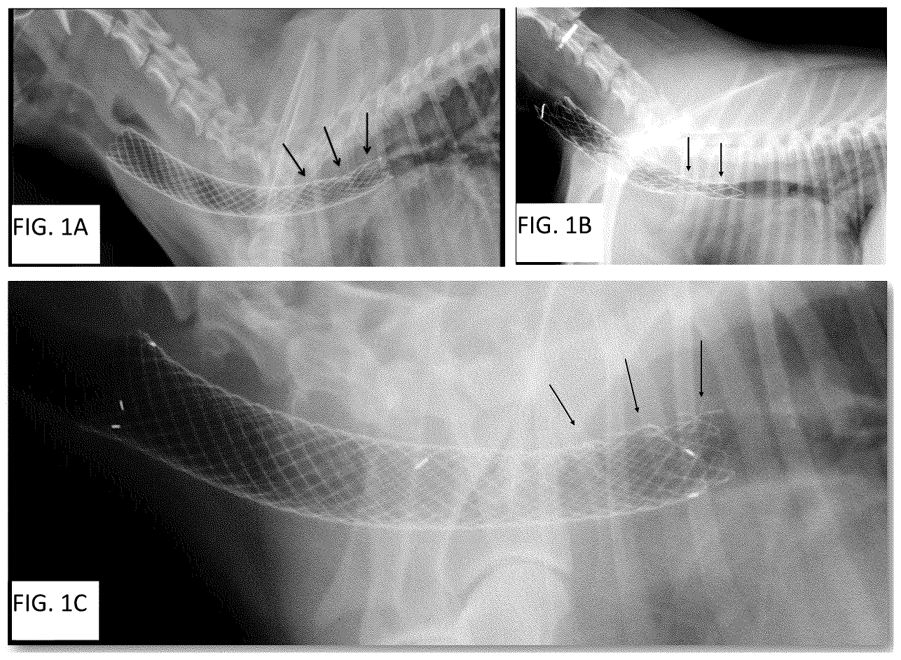

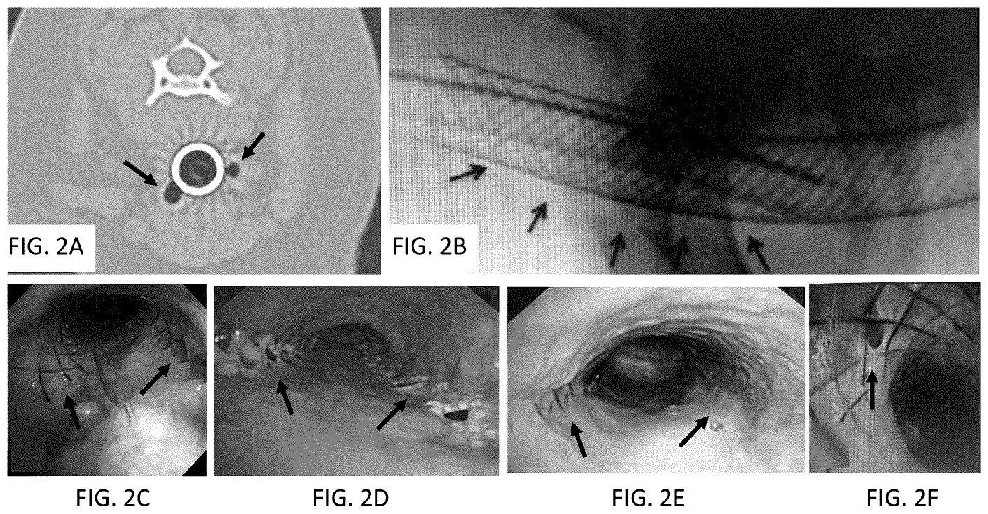

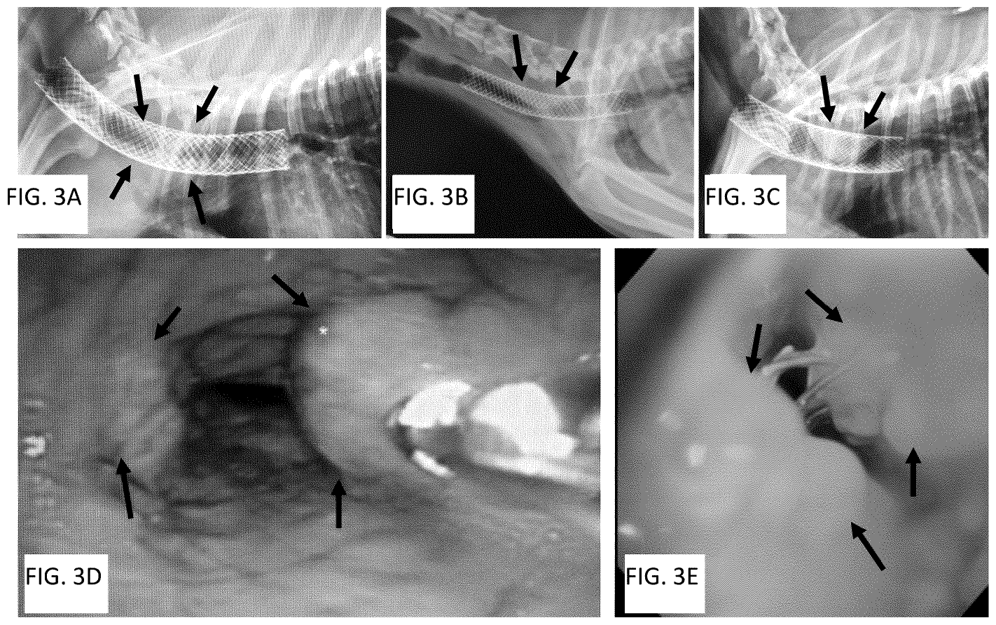

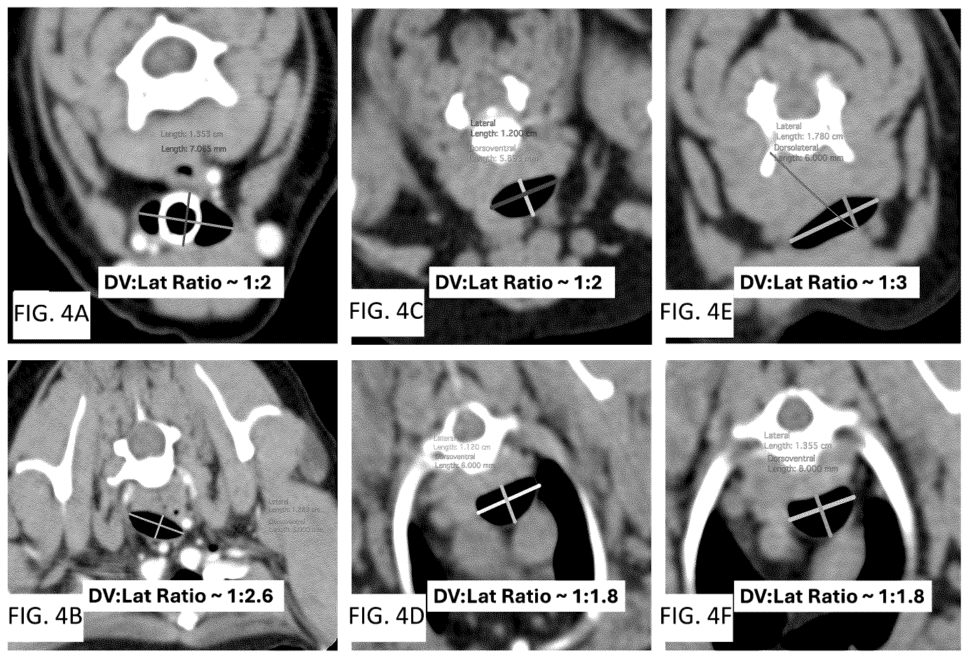

Regardless of the material (metal, plastic, or other) from which the stent is manufactured or the design (laser-cut, wire wound, bare or covered, etc.) of the airway stent, oversizing of the stent will be responsible for maintaining continuous circumferential contact with the tracheobronchial wall to limit migration (and further foreshortening for wire-wound designs). The more conformal shape of an airway stent intended for a canine collapsed trachea allows for less oversizing of the airway stent to reduce oversizing complications commonly encountered with round airway stents that lead to stent cycling, fatigue, and fracture (FIGS. 1A-1C). Additionally, the airway stent provides improved stent-to-tracheobronchial wall contact, reducing the likelihood of gaps (FIGS. 2A through 2F) leading to mucus accumulation and trapping, chronic infections, tissue ingrowth, and airway occlusion (FIGS. 3A through 3E) that can lead to the requirement for additional procedures or lead to patient respiratory distress or death.

From the above, it should be appreciated that the present invention provides for facile implantation of the airway stent within a patient to provide a more anatomically appropriate device reducing the limitations encountered with the currently available round airway stents. The airway stent provides the capability for easy placement in the commonly encountered, non-uniform shaped tracheobronchial trees of dogs suffering from CTCS. The variety of sizes and delivery system options, as well as the reconstrainable and repositionable nature of the airway stent designs provide safe, non-painful, non-invasive, and effective anatomically appropriate placement of the airway stent without the requirement for risky, open surgery, or problems with stent oversizing and gaps currently leading to the most common complications seen with round airway stents. Patients with a variety of tracheobronchial airway obstructions (collapse, stenosis, benign or malignant tissue obstruction, etc.) may benefit from the airway stent.

The most common complications reported for currently available round airway stents include infection (approximately 66%), tissue ingrowth (approximately 19-38%), and stent fracture (approximately 25%). There are considerable advantages of the airway stent represented in FIGS. 5A through 5D over such devices, including but not limited to the following issues encountered with prior art stents.

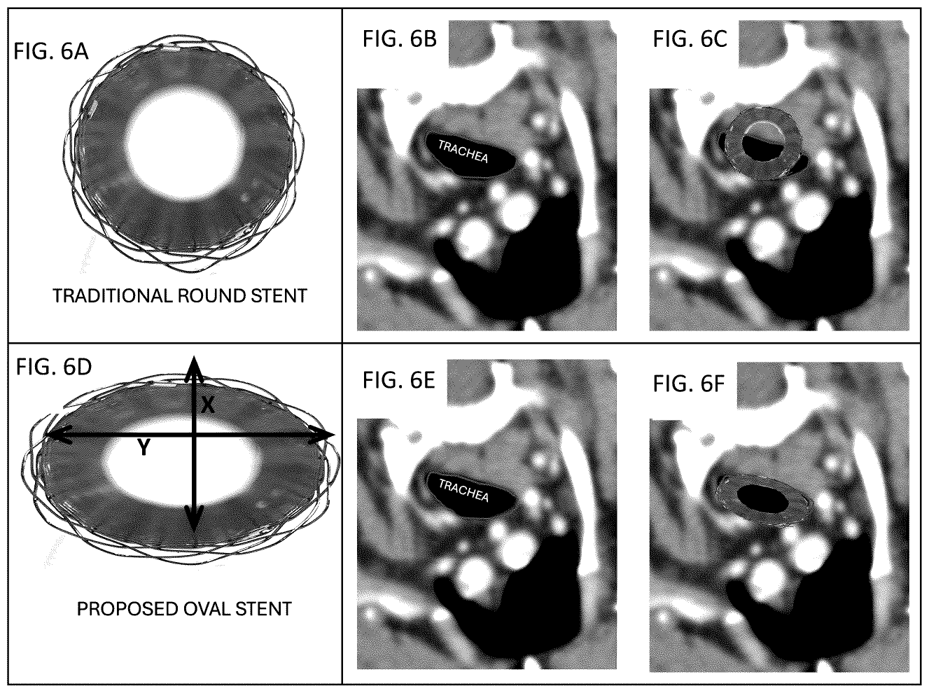

Infection: Infection is a common problem in dogs suffering from CTCS, even prior to stent placement. Approximately 66% of CTCS patients will have current, active tracheobronchial infections at the time of stent placement. These infections can be cleared once the airway obstructions have been repaired and the mucociliary apparatus is restored. This is most successful in older patients with traditional types of CTCS. The patients in whom tracheal infections become chronic tend to be those dogs with stents that have incomplete stent-to-tracheobronchial wall contact and gaps (FIGS. 2A-2F, 3A-3E, and 6A-6C). Gaps resulting from round stents permit mucus trapping and the inability of the patient to expectorate and clear mucus and pus. The patients remain at increased risk for chronic infections with bacterial resistance. Airway stents disclosed herein solve this issue by providing more anatomically appropriate stent-to-tracheobronchial wall contact, limiting any gaps through complete stent endothelialization in the tracheal wall, re-establishment of the mucociliary apparatus, and proper mucus clearance.

Tissue ingrowth: Tissue ingrowth following airway stent placement has several underlying causes including infection, micromotion, and possible stent material sensitivities, etc. The latter two of these possible causes are difficult to control with current stent technology. The first cause—infection—can be limited by reducing tracheobronchial infections. Because the gaps caused by round stents in naturally oval CTCS airways are unavoidable in many cases, tissue ingrowth (and infection) remain common post-stent complications. Dimensions of airway stents as disclosed herein are based on numerous measurements in a population of dogs suffering from CTCS (Weisse unpublished data, 2024). This data clearly demonstrated the oval or elliptical (not circular) cross-sections of the CTCS airways and the limitations of prior art round airway stents. The airway stents disclosed herein provide improved stent-to-tracheobronchial wall contact. This permits complete stent endothelialization without the gaps that currently lead to mucus trapping, infection, and the life-threatening tissue ingrowth and airway obstruction that often ensue.

Fracture: Stent fractures occur in approximately 25% (or more) of canine patients following airway stenting with the currently available round airway stents available (Violette et al. 2019). It seems this occurs with any type of stent (wire-wound or laser-cut design) used in these patients. Previous research suggests stent fracture is significantly more likely in patients with a naturally tapering tracheal diameter, and those patients with greater stent oversizing in the intrathoracic trachea (Violette et al. 2019). Because of the clear problems associated with incomplete round stent-to-tracheobronchial wall contact, clinicians purposely select round airway stents with oversized diameters to limit the possible resulting gaps; however, this increases the patient's risk of subsequent stent fracture. Fractured stents cannot be easily or safely removed so these patients require another procedure to place a second airway stent which also remains at risk for fracture. The airway stents disclosed herein can be configured to fit within the naturally oval tracheobronchial cross section of a CTCS airway more precisely, and therefore do not need to be so dramatically oversized as currently-available round airway stents. Based on previous studies in stented CTCS patients, the airway stent will reduce the likelihood of subsequent stent fracture.

Technical publications that relate to the present disclosure are listed below, and the contents of these publications are incorporated herein by reference. The publications are being cited herein with the intent that they may help facilitate a better understanding of the disclosure, and their citation is not to be construed as an admission of prior art or what is or is not relevant prior art to the present invention.

While the invention has been described in terms of particular but nonlimiting embodiments, it is apparent that other forms could be adopted by one skilled in the art. For example, the airway stent could differ in appearance and construction from the embodiments disclosed, each component of the airway stent could be performed by components of different construction but capable of a similar (though not necessarily equivalent) function, and appropriate materials could be substituted for those noted. As such, and again as was previously noted, it should be understood that the invention is not necessarily limited to any particular embodiment described herein or illustrated in the drawings.

1. A procedure for placing an airway stent for treating large airway (trachea and bronchus) obstructions in which the airway cross-section is oval or elliptical, the procedure comprising:
providing an airway stent that has an oval or elliptical (not circular) cross-sectional shape;
placing the airway stent in the airway through a delivery system comprising an endotracheal tube preplaced in a patient;
ensuring positioning of the airway stent via delivery system markers indicating dorsoventral (anterior-posterior) positioning;
partially deploying the airway stent under fluoroscopic guidance;
repositioning the airway stent as needed through delivery system reconstrainment;
completely deploying the airway stent so that the airway is patent; and
removing the delivery system through the airway.

2. The procedure according to claim 1, wherein the airway stent is manually mounted onto a delivery system or placed without prior delivery system mounting.

3. The procedure according to claim 1, wherein the delivery system is passed directly through the glottis, a tracheotomy, or other open surgical approach.

4. The procedure according to claim 1, wherein stent deployment is guided using radiography, endoscopy, CT, or open surgical visualization.

5. The procedure according to claim 1, wherein the airway stent is secured in place using suture or toggles rather than by solely oversizing the airway stent to maintain position within the airway.

6. The procedure according to claim 1, wherein the patient is a child or an adult human.

7. The procedure according to claim 1, wherein the patient is an animal.

8. The procedure according to claim 1, wherein the patient is a dog suffering from CTCS.

9. The procedure according to claim 1, wherein the airway stent remains temporary or remains indwelling long-term within the patient.

10. An airway stent for placement in an airway, the airway stent comprising a cross-sectional shape such that the airway stent has a dorsoventral (anterior-posterior) diameter that is different from a lateral diameter of the airway stent, wherein the airway stent has a dorsoventral:lateral ratio of about 1:1.5 to about 1:3.

11. The airway stent of claim 10, wherein the dorsoventral diameter is about 6 mm to about 20 mm.

12. The airway stent of claim 11, wherein the lateral diameter is about 8 mm to about 60 mm.

13. The airway stent of claim 10, wherein the dorsoventral diameter, the lateral diameter, and the dorsoventral:lateral ratio varies along a length of the airway stent such that the dorsoventral:lateral ratio is not constant along the length.

14. The airway stent of claim 10, wherein the airway stent has a length of about 15 mm to about 150 mm.

15. The airway stent of claim 10, wherein the airway stent is bare, partially covered, or fully covered.

16. The airway stent of claim 10, wherein the airway stent is formed of metal, plastic, or other flexible biocompatible material.

17. The airway stent of claim 10, wherein the airway stent has a dorsoventral diameter, a lateral diameter, and a dorsoventral:lateral ratio determined through serial CTCS large airway imaging studies to provide a conformal anatomical compliance of the airway stent within an oval-shaped airway.

18. The airway stent of claim 10, wherein the airway stent has a dorsoventral diameter, a lateral diameter, and a dorsoventral:lateral ratio that reduce oversizing needed for securing the airway stent within the airway and thereby reduce stent fatigue and subsequent fracture.

19. The airway stent of claim 10, wherein the airway stent has a dorsoventral diameter, a lateral diameter, and a dorsoventral:lateral ratio that promotes stent-to-airway wall contact, reduces stent oversizing relative to the airway, improves stent endothelialization within the airway, and reduces gaps between the airway stent and the airway so as to reduce mucus accumulation.
